(19) United States
(12) Patent Application Publication
(10) Pub. No.: US 2011/0196472 A1
(43) Pub. Date: Aug. 11, 2011

(54) STENT DELIVERY SYSTEM

(75) Inventors: Ryota SUGIMOTO, Ashigarakami-gun (JP); Takashi Kitaokia, Ashigarakami-gun (JP)

(73) Assignee: Terumo Kabushiki Kaisha, Tokyo (JP)

(21) Appl. No.: 13/074,519

(22) Filed: Mar. 29, 2011

Related U.S. Application Data
(63) Continuation of application No. PCT/JP2009/066448, filed on Sep. 18, 2009.

(30) Foreign Application Priority Data
Sep. 30, 2008 (JP) 2008-254742

(51) Int. Cl. A61F 2/82

(52) U.S. Cl. 623/1.12

(57) ABSTRACT

A stent delivery system includes a self-expandable stent, a shaft section having a guide wire lumen, and a sheath having distal portion containing the stent. The stent is located at a position which is on the shaft section and near the distal end of the shaft section. The stent delivery system has a stent proximal portion fixing wire and a breaking member. The stent proximal portion fixing wire has one end portion and the other end portion which are fixed to the shaft section, and also has an intermediate portion engaged with a proximal portion of the stent. The breaking member breaks the stent proximal portion fixing wire to release the stent from the engagement.

This application is a continuation of International Application No. PCT/JP2009/066448 filed on Sep. 18, 2009, and claims priority to Japanese Application No. 2008-254742 filed on Sep. 30, 2008, the entire content of both of which is incorporated herein by reference.

### TECHNICAL FIELD

The present invention relates to a stent delivery system for use in improvement of a stenosed or occluded lesion generated in a living body lumen such as blood vessel, bile duct, trachea, esophagus, urethra, etc.

### BACKGROUND DISCUSSION

A stent for placement (indwelling) in living bodies is generally a tubular medical device which, for treatment of various diseases generated by stenosis or occlusion of a blood vessel or other living body lumen, is placed (put indwelling) in the stenosed or occluded lesion so as to dilate the lesion and secure the lumen (maintain the lumen in an open state) at the lesion.

The following description describes a non-limiting example in which the stent is used in a blood vessel.

The stent is a body which, for insertion from the outside into the inside of a living body, is small in diameter at the time of insertion, and is outwardly expanded at the target region (the stenosed or occluded lesion) so as to be enlarged in diameter and to maintain the lumen at the lesion in an open state.

In general, stents are cylindrical bodies obtained by processing metallic wires or metallic pipe. A stent is mounted to a catheter or the like in a radially reduced state, is inserted into a living body, and is expanded at a target site by an appropriate method to be fixed in close contact with the inner wall of the lumen at the lesion, thereby maintaining the lumen shape. Stents are oftentimes classified by function and placement method into self-expandable stents and balloon-expandable stents. A balloon-expandable stent is a stent which itself does not have a self-expanding function or capability. The balloon-expandable stent is mounted on a balloon, is inserted into a target lesion, and thereafter the balloon is dilated of inflated to outwardly expand (plastically deform) the stent by the expanding force of the balloon, thereby fixing the stent in close contact with the inner surface of the target lumen. This type of stent requires the stent-expanding operation as above-mentioned. On the other hand, a self-expandable stent is a stent which itself has a self-expanding function (i.e., is capable of expanding on its own). The use of the self-expandable stent involves inserting the self-expandable stent in a radially contracted state into a living body, and releasing the stent from the contracted state at a target region (target lesion) to return the stent to its original outwardly expanded state. This fixes the stent in close contact with the inner wall of the lumen at the lesion and maintains the lumen shape (i.e., maintains the open state of the lumen).

The purpose of the placement of a stent at present is to return a blood vessel stenosed for some reason to its original open state. In most cases, the stents are mainly for preventing or reducing the risk or extent of restenosis which might occur after a procedure such as PTCA. In recent years, to more reliably suppress the probability of restenosis, drug-eluting stents coated with a drug such as immunosuppressor or carcinostatic have been used.

Most of the self-expandable stents are used in peripheral regions such as inferior-limb blood vessels and carotid arteries. There are self-expandable stents which have, for example, the form shown in JP-T Hei 11-505441,

In the stent delivery system using a self-expandable stent as disclosed in JP-T Hei 11-505441, the self-expanding property of the stent makes it difficult to properly position the stent at the time of placement of the stent, as compared with a balloon-expandable stent. In addition, there may arise a jumping phenomenon in which the stent jumps out of the delivery system. If this phenomenon occurs, the stent would be arranged at a position deviated from a planned arrangement position. In a stent placing procedure, re-adjustment of the placing position may in some cases be needed after the stent is discharged to a certain extent. In such cases as described in JP-T Hei 11-505441, however, it is difficult to re-contain the stent into the stent delivery system.

### SUMMARY

As disclosed here, a stent delivery system uses a self-expandable stent and is configured so that the stent is not so likely to exhibit a jumping action associated with the self-expanding property of the stent jump during deployment of the stent, thus facilitating placement of stent at the desired place. The stent delivery system is also configured so that during deployment of the stent, the stent can be once again contained in the stent delivery system.

A stent delivery system disclosed here includes: a cylindrically shaped stent (inclusive of substantially cylindrical in shape) which is compressed toward a central axis at the time of insertion into a living body, and is configured to expand outwardly when placed in the living body to be restored to a pre-compression shape; a shaft section having a guide wire lumen; and a sheath having a distal portion in which is contained the stent. The stent is located at a position which is on the shaft section and near the distal end of the shaft section; wherein the stent delivery system includes a stent proximal portion fixing wire having one end portion and the other end portion fixed to the shaft section, and having an intermediate portion engaged with a proximal portion of the stent, and a breaking member for breaking the stent proximal portion fixing wire to release the stent from the engagement.

This configuration of the stent delivery system effects engagement of the proximal portion of the stent with the shaft section until the stent proximal portion fixing wire is broken. The stent is thus not susceptible to jumping out during discharge of the stent from the sheath. In addition, even after the discharge of the stent from the sheath is initiated, the stent can be re-contained in the sheath if the stent proximal portion fixing wire is not yet broken. Accordingly, the position of the stent can be corrected, thus facilitating correct positioning of the stent at a target site.

If the stent proximal portion fixing wire extends in the direction of the stent by passing one end portion through the gap in the coil constituting the spring-formed stopper, with the other end portion fixed to the shaft section, the position of the wire is stabilized. Fixation of the stent by the wire is relatively assured, and the wire can be released from the stent favorably.

The proximal portion of the stent can be provided with a plurality of through holes through which passes the stent proximal portion fixing wire. The holes can be arranged in a substantially annular pattern, with the intermediate portion of the stent proximal portion fixing wire passing annularly through the plurality of holes in the stent. The through holes can be configured to include a low-friction inner surface or a relatively easily releasable form for enhancing releasability of the wire.

The proximal portion of the stent preferably has a plurality of proximal direction bent portions, and the intermediate portion of the stent proximal portion fixing wire passes annularly through the plurality of proximal direction bent portions of the stent.

The shaft section has preferably a distal tube having the guide wire lumen, and a shaft body having a distal portion fixed to the proximal side of the distal tube, and the breaking member is provided at the distal portion of the shaft body.

According to one embodiment, the stent has a distal portion oriented toward the distal side of the sheath and a proximal portion oriented toward the proximal side of the sheath. The stent preferably does not have any bent free end at least projecting toward the proximal side, exclusively of the proximal portion. The stent delivery system is preferably configured such that after exposing the distal portion of the stent from the sheath, the exposed distal portion can be re-contained into the sheath by moving the sheath.

In the disclosed embodiment, the stent proximal portion fixing wire is a heat-breaking stent proximal portion fixing wire, and the breaking member is a heat-breaking member. The heat-breaking member can include a heat generating section for breaking, an electric cable having a distal portion connected to the heat generating section and extending toward a proximal portion of the shaft body, and a joint section for joining to a power supply section. The joint section can be connected to the electric cable and formed at the proximal portion of the shaft body.

The shaft section preferably has a proximal-side opening communicating with the guide wire lumen. The proximal-side opening opens at a side portion on the proximal side relative to a stent-containing part of the sheath. The sheath also has a sheath side hole on the proximal side relative to the stent-containing part, and the guide wire is insertable through the sheath side hole and the proximal-side opening.

The stent delivery system is preferably configured so that the stent can be re-contained into the sheath until the stent is released from the engagement through breaking of the stent proximal portion fixing wire. The stent proximal portion fixing wire can be a thermoplastic resin fiber.

The shaft section has a proximal-side stopper located near the proximal end of a part where the stent is disposed and which restrains the stent from moving in the proximal direction. The proximal-side stopper is a spring-formed stopper wound around the shaft section.

The stent proximal portion fixing wire preferably extends in the direction of the stent by passing through a gap in a coil constituting the spring-formed stopper, from one end portion and the other end portion which are fixed to the shaft section.

The stent preferably does not have any free end, due to a structure in which an apex or a portion near the apex, of a proximal-side bent portion, is joined to another linear element. In the illustrated embodiment, the stent includes a plurality of wave-shaped struts extending in the axial direction of the stent from one end side to the other end side of the stent and arranged in the circumferential direction of the stent. In addition, one or more connecting struts interconnect the wave-shaped struts which are adjacent to each other, with the connecting struts extending in the axial direction over a predetermined length. The wave-shaped struts each have end portions each joined to an end portion of the wave-shaped strut close thereto. The connecting struts can be curved in a circular arc shape.

The stent can also include joint sections for joining each of one-end-side end portion and an the-other-end-side end portion of each wave-shaped strut to an end portion of another wave-shaped strut close to the each wave-shaped strut, and the joint section on one-end-side and the joint section on the-other-end-side differ from each other in combination of the wave-shaped struts which are joined to each other.

### BRIEF DESCRIPTION OF THE DRAWING FIGURES

FIG. 1 is a front view of a stent delivery system according to an embodiment disclosed here.

FIG. 2 is a longitudinal cross-sectional view of the stent delivery system shown in FIG. 1.

FIG. 3 is a front view of the sheath in the stent delivery system shown in FIG. 1.

FIG. 4 is a front view of a shaft section in the stent delivery system shown in FIG. 1.

FIG. 5 is an enlarged longitudinal cross-sectional view of a distal portion of the stent delivery system shown in FIG. 1.

FIG. 6 is an enlarged longitudinal cross-sectional view of an intermediate portion of the stent delivery system shown in FIG. 1.

FIG. 7 is an enlarged longitudinal cross-sectional view of a proximal portion of the sheath in the stent delivery system shown in FIG. 1.

FIG. 8 is an enlarged longitudinal cross-sectional view of a proximal portion of the shaft in the stent delivery system shown in FIG. 1.

FIG. 9 is an illustration of a proximal portion of a stent in the stent delivery system shown in FIG. 1.

FIG. 10 is a front view of an example of a stent for placement in living body used in the stent delivery system disclosed here.

FIG. 11 is a development view of the stent shown in FIG. 10.

FIG. 12 is an enlarged view of a through hole in a proximal portion of the stent shown in FIG. 10.

FIG. 13 is an enlarged cross-sectional view taken along the section line XIII-XIII in FIG. 12.

FIG. 14 is an enlarged longitudinal cross-sectional view of a distal portion in the stent delivery system according to another embodiment disclosed here.

FIG. 15 is an enlarged perspective view of a through hole in a proximal portion of the stent used in the stent delivery system disclosed here.

FIG. 16 is an illustration of the operation of the stent delivery system disclosed here.

FIG. 17 is an illustration of the operation of the stent delivery system disclosed here.

FIG. 18 is an illustration of the operation of the stent delivery system disclosed here.

### DETAILED DESCRIPTION

Set forth below is a description of the stent delivery system disclosed here. The stent delivery system constitutes a living organ dilator.

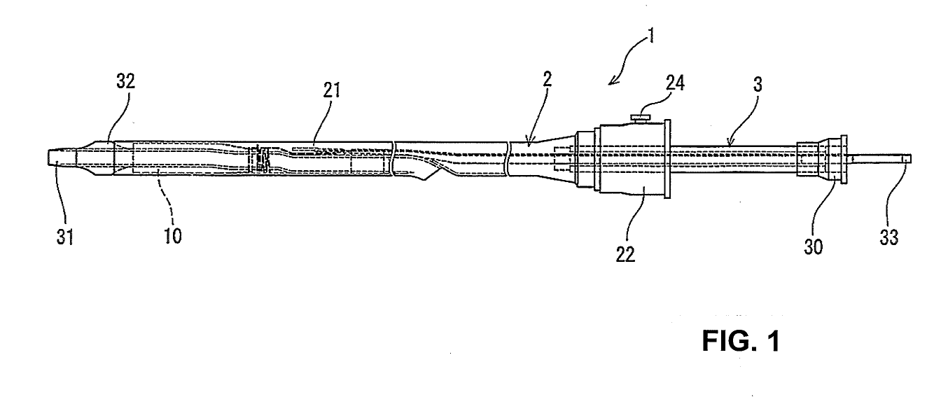

Referring initially to FIG. 1, the stent delivery system 1 includes: a stent 10 which is cylindrical (inclusive of substantially cylindrical) in shape, compressed radially inwardly toward its center axis at the time of insertion into a living body, and capable of expanding (automatically expanding) radially outwardly when placed (put indwelling) in the living body, to be restored to its pre-compression shape; a shaft section 3 having a guide wire lumen 61; and a sheath 2 having a distal portion in which is contained the stent 10, with the stent 10 being positionally located at the shaft section 3 and near the distal end of the shaft section 3. Further, the stent delivery system 1 includes a stent proximal portion fixing wire 5 (fixing wire) having one end portion 5a, an other end portion 5b fixed to the shaft section 3, and an intermediate portion 5c engaged with a proximal portion of the stent 10, and a breaking member 7 for breaking the stent proximal portion fixing wire 5 to release the stent from engagement with the stent proximal portion fixing wire 5.

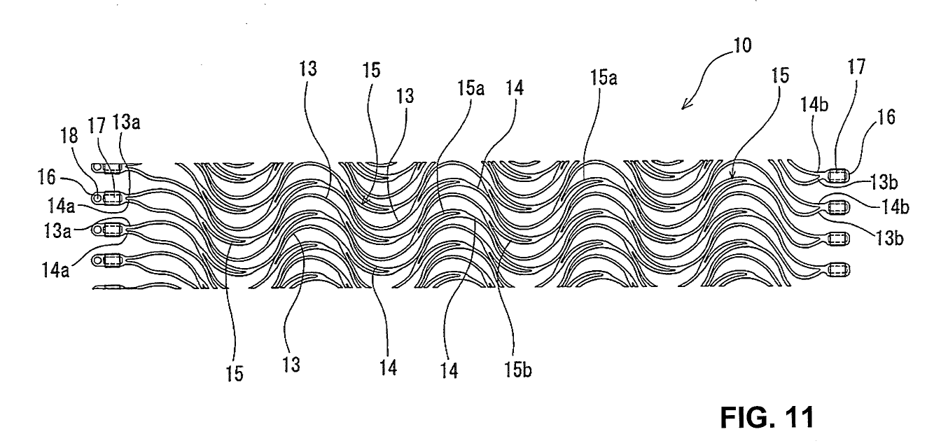

In the embodiment of the stent delivery system 1 shown in the drawings, the stent 10 is configured to expand radially outward after being placed (put indwelling) in a living body and to be restored into its pre-compression shape, the stent is contained in the distal portion of the sheath 2, and the shaft section 3 is slidably inserted in the sheath 2 and is configured to discharge the stent 10 through the distal end of the sheath 2. The stent 10 used here has a distal portion oriented toward the distal side of the sheath 2 and a proximal portion oriented toward the proximal side of the sheath 2. In addition, other than the proximal end portion of the stent 10 at which the proximal-most end portions 13a, 14a are joined together at the joint portions 16 as shown in FIG. 11, the stent 10 is devoid of bent free ends projecting toward the proximal side (in the proximal direction). In addition, by moving the sheath 2 after exposure of a distal portion of the stent 10 from the sheath 2, the exposed distal portion can be re-contained into the sheath 2. The stent delivery system 1 includes the guide wire lumen 61 having one end opening at the distal end of the stent delivery system, and the other end opening at the proximal side relative to the stent-containing part of the sheath 2. The shaft section 3 of the illustrated embodiment of the stent delivery system 1 includes the stent proximal portion fixing wire 5 having the one end portion 5a fixed to the shaft section, the opposite end portion 5b fixed to the shaft section 3 and the intermediate portion 5c engaged with a proximal portion of the stent 10, and the breaking member 7 for breaking the stent proximal portion fixing wire 5 to release the stent 10 from the engagement with the stent proximal portion fixing wire 5.

The stent delivery system 1 is composed of the stent 10, the sheath 2 having the stent 10 contained in its distal portion, and the shaft section 3 slidably inserted in the sheath 2.

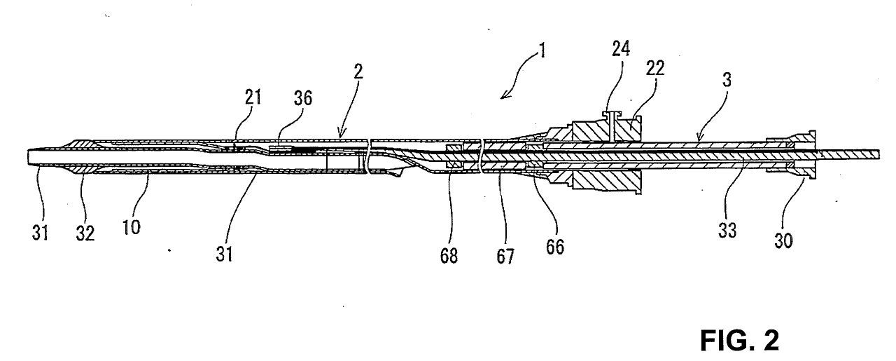

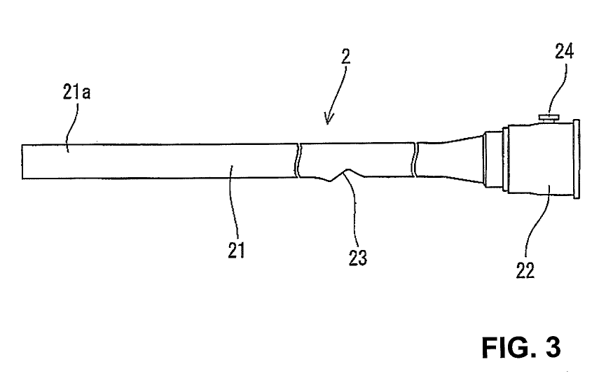

As shown in FIGS. 1 to 7, the sheath 2 includes a sheath tube 21, and a sheath hub 22 fixed to the proximal end of the sheath tube 21.

The sheath tube 21 is a tubular body which is open at both at its front end (tip) and its rear end. The tip opening functions as a discharge port for the stent 10 when the stent 10 is placed (put indwelling) in a stenosed lesion in a body lumen. By being pushed out via the tip opening, the stent 10 is released from a stress load, to expand and to be restored into its pre-compression shape. The distal portion of the sheath tube 21 constitutes a stent-containing part 21a for containing the stent 10. In addition, the sheath tube 21 has a side hole 23 proximally located relative to the stent-containing part 21a. The side hole 23 is for leading out a guide wire to the exterior.

The outside diameter of the sheath tube 21 is preferably about 0.5 to 4.0 mm, more preferably 0.8 to 2.0 mm. The inside diameter of the sheath tube 21 is preferably about 0.2 to 1.8 mm. The length of the sheath tube 21 is preferably 300 to 2500 mm, more preferably about 300 to 2000 mm.

The material forming the sheath tube 21 is appropriately selected while taking into account the physical properties required of the sheath tube (flexibility, hardness, strength, slidability, anti-kinking property, and expansion/contraction properties). Preferably, the material is selected, for example, from among polyethylene, polypropylene, nylon, polyethylene terephthalate, fluoro-polymers such as PTFE, ETFE, etc. and thermoplastic elastomers. The thermoplastic elastomers are appropriately selected from among nylon-based ones (e.g., polyamide elastomer), urethane-based ones (e.g., polyurethane elastomer), polyester-based ones (e.g., polyethylene terephthalate elastomer), and olefin-based ones (e.g., polyethylene elastomer, polypropylene elastomer).

The outer surface of the sheath tube 21 is preferably subjected to a treatment rendering the outer surface lubricious. Examples of such a treatment include coating the outer surface with a hydrophilic polymer or fixing a hydrophilic polymer to the outer surface. Examples of the hydrophilic polymer include poly(2-hydroxyethyl methacrylate), polyhydroxyethyl acrylate, hydroxypropyl cellulose, methyl vinyl ether-maleic anhydride copolymer, polyethylene glycol, polyacrylamide, polyvinyl pyrrolidone, etc. In addition, the inner surface of the sheath tube 21 may be coated with the above-mentioned hydrophilic polymer or the hydrophilic polymer may be fixed to the inner surface, in order to enhance slidability between the inner surface and the stent 10 as well as the shaft section 3.

As shown in FIGS. 1-3 and 7, a sheath hub 22 is fixed to a proximal portion of the sheath tube 21. FIG. 7 illustrates that a seal member 25 is positioned between the inner surface of the sheath hub 2 and the outer surface of the shaft section 3 to holds the shaft section 3 in a slidable and liquid-tight manner. The sheath hub 22 has a side port 24.

The material forming the sheath hub 22 is preferably a hard or semi-hard material. Examples of the hard or semi-hard material which can be used here include synthetic resins such as polycarbonate, polyolefins (e.g., polyethylene, polypropylene, ethylene-propylene copolymer), styrene reins [e.g., polystyrene, MS resin (methacrylate-styrene copolymer), MBS resin (methacrylate-butylene-styrene copolymer)], polyesters, etc., and metal such as stainless steel, aluminum, aluminum alloys, etc.

The material forming the seal member 25, and an elastic ring 69 to be described later, is preferably an elastic material. Examples of the elastic material include rubbers such as synthetic rubbers such as urethane rubber, silicone rubber, butadiene rubber, etc. and natural rubbers such as latex rubber, etc., and synthetic resin elastomers such as olefin elastomers (e.g., polyethylene elastomer, polypropylene elastomer), polyamide elastomers, styrene elastomers (e.g., styrene-butadiene-styrene copolymer, styrene-isoprene-styrene copolymer, styrene-ethylenebutylene-styrene copolymer), polyurethane, urethane elastomers, fluoro-resin elastomers, etc.

Reinforcement members 26, 27 are provided at a distal portion of the sheath hub 22. These reinforcement members 26, 27 extend in the distal direction from the distal end of the sheath hub.

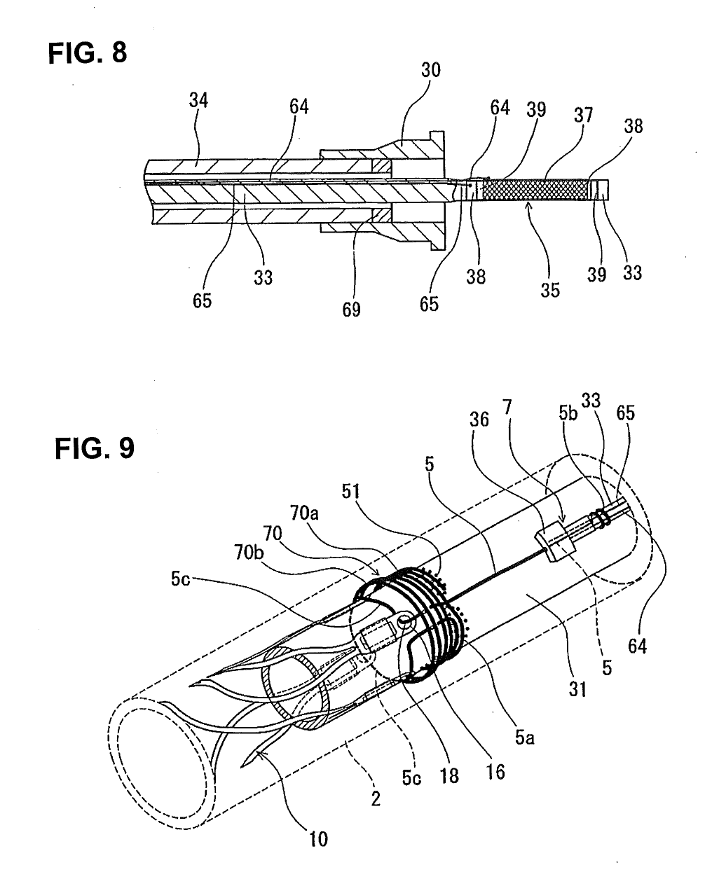

As shown in FIGS. 1-8, the shaft section 3 includes: a shaft body 33; a distal tube 31 provided at the distal end of the shaft body 33 and protruding distally beyond the distal end of the sheath 2; a shaft hub 30 fixed to a proximal portion of the shaft body 33; a stent proximal portion fixing wire 5 fixed to the shaft body 33; and a breaking member or breaking means 7 for breaking the stent proximal portion fixing wire 5. In the illustrated arrangement, the breaking means is provided on the shaft body 33.

In this embodiment, the stent proximal portion fixing wire 5 is a heat-breaking stent proximal portion fixing wire (i.e., a fixing wire that is breakable upon being heated), and the breaking means or breaking member 7 is a heat-breaking member (i.e., a unit that breaks the fixing wire through application of heat). But the stent proximal portion fixing wire 5 and the breaking member 7 are not limited in this manner. The stent proximal portion fixing wire 5 and the breaking member 7 may be configured to break the stent electrically, mechanically, or by water pressure or the like, thereby being released from the shaft section 3.

In this embodiment, the shaft section 3 has a proximal-side opening of the guide wire lumen opening at a side part on the proximal side relative to the stent-containing part of the sheath 2, whereas the sheath 2 has a sheath side hole provided on the proximal side relative to the stent-containing part, and a guide wire can be inserted through the sheath side hole and the proximal-side opening.

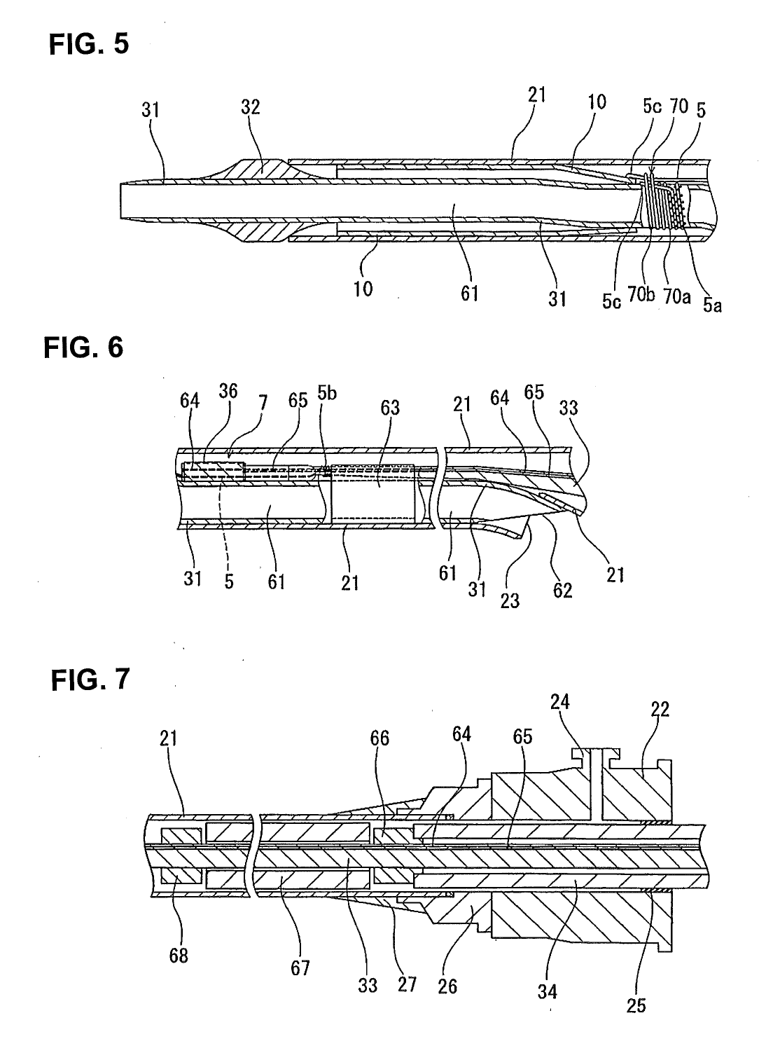

As shown in FIG. 5, the distal tube 31 protrudes distally beyond the distal end of the sheath 2. In addition, the distal tube 31 is provided with a stopper 32 for inhibiting the sheath 2 from moving in the distal direction. As shown in FIG. 6, a proximal portion of the distal tube 31 is curved, enters into a side hole 23 of the sheath tube 21, and disengageably engages the side hole 23. The outside diameter of the distal tube 31 is preferably 0.2 to 1.8 mm. The distal portion of the distal-side stopper 32 is preferably reduced in diameter toward the distal direction as illustrated in FIG. 5. The outside diameter of a maximum-diameter portion of the stopper 32 is preferably 0.5 to 4.0 mm. In addition, a proximal portion of the stopper 32 is also preferably reduced in diameter toward the proximal direction as shown in FIG. 5. The guide wire lumen 61 in the distal tube 31 extends from the distal end of the distal tube 31 to the proximal end of the distal tube 31. The proximal opening 62 of the guide wire lumen 61 is preferably proximally spaced from the distal-most end of the distal tube 31 by 10 to 400 mm, more preferably 50 to 350 mm. In addition, the proximal opening 62 is preferably spaced in the proximal direct from the rear end (proximal-most end) of the stent 10 (in other words, the rear end of the stent-containing part) by about 50 to 250 mm.

The shaft body 33 has a distal section fixed to a proximal portion of the distal tube 31, a body section extending proximally over a predetermined distance, and a proximal section protruding proximally beyond the proximal end of the shaft hub 30. In this embodiment, the shaft body 33 is configured such that the distal portion of the shaft body 33 fixed to the distal tube 31 is a small-diameter section, and the body section and the proximal section possess outside diameters greater than the small-diameter section. In this embodiment, the distal section of the shaft body 33 is fixed to a side surface of the distal tube 31 through a heat-shrinking tube 63.

The length of the shaft section 3 is preferably about 400 to 2500 mm, more preferably 400 to 2200 mm. In addition, the outside diameter of the body section of the shaft body 33 is preferably about 1.0 to 2.5 mm, more preferably 1.0 to 2.0 mm. The length of the distal tube 31 is preferably about 10 to 400 mm, more preferably 50 to 350 mm, and the outside diameter of the distal tube 31 is preferably about 0.2 to 2.0 mm. In addition, the inside diameter of the lumen 61 is preferably about 0.2 to 2.0 mm, more preferably 0.3 to 1.0 mm.

The shaft body 33 may be either solid or tubular. In addition, it may be a coil shaft. The material forming the shaft section 3 is preferably a material which has hardness and a certain degree of flexibility. Preferable examples of the material include metallic wires or metallic pipes of stainless steel, superelastic metals and the like, and bar-like bodies or annular bodies or the like of polyethylene, polypropylene, nylon, polyethylene terephthalate, fluoro-polymers such as ETFE, etc., PEEK (polyether ether ketone), polyimide, etc. Incidentally, an outer surface of the shaft section 3 may be coated with a resin having bio-compatibility, particularly, anti-thrombotic property. Examples of the anti-thrombotic material which can be preferably used here include polyhydroxyethyl methacrylate, copolymers of hydroxyethyl methacrylate and styrene (e.g., HEMA-St-HEMA block copolymer), etc.

Further, the outer surface of that portion of the shaft section 3 which protrudes from the sheath 2, preferably has lubricity. In view of this, the outer surface may be coated with a hydrophilic polymer or a hydrophilic polymer may be fixed to the outer surface. Examples of the hydrophilic polymer include poly(2-hydroxyethyl methacrylate), polyhydroxyethyl acrylate, hydroxypropyl cellulose, methyl vinyl ether-maleic anhydride copolymer, polyethylene glycol, polyacrylamide, polyvinyl pyrrolidone, etc. In addition, the entirety of the outer surface of the shaft section 3 may be coated with the above-mentioned hydrophilic polymer or the hydrophilic polymer may be fixed to the entire outer surface. Furthermore, the inner surface of the shaft section 3 may be coated with the above-mentioned hydrophilic polymer or the hydrophilic polymer may be fixed to the inner surface, in order to enhance slidability between the inner surface and a guide wire.

The shaft body 33 penetrates the sheath 2, and protrudes proximally from the rear-end opening of the sheath 2. As shown in FIGS. 1-3 and 8, the shaft hub 30 is firmly attached to a proximal portion of the shaft body 33. In this embodiment, a fixation ring 66 is fixed to the shaft body 33 as shown in FIG. 7. In addition, a proximal tube 34 is fixed to the shaft hub 30 and extends in the distal direction from the shaft hub 30. A distal portion of the proximal tube 34 is fixed to the fixation ring 66. An elastic ring 69 is fixed to the proximal end of the proximal tube 34 (in the inside of the shaft hub 30). In this embodiment, a second fixation ring 68 is distally spaced a predetermined distance from the fixation ring 66, and an intermediate tube 67 is disposed between the first fixation ring 66 and the second fixation ring 68. The intermediate tube 67 is fixed to neither the shaft body 33 nor the sheath tube 21, and is adapted to abut both the first fixation ring 66 and the second fixation ring 68. The intermediate tube helps promote favorable sliding of the sheath. The intermediate tube 67 is preferably a tube which has a low-friction surface. Specifically, a tube formed of, for example, polyethylene, polypropylene, nylon, polyethylene terephthalate, a fluoro-polymers such as PTFE and ETFE, or the like is preferably used.

As shown in FIGS. 5 and 9, a proximal-side stopper 70 for restraining the stent 10 from moving in the proximal direction is provided at a distal portion of the shaft section 3 (specifically, near the proximal end of that part of the distal tube 31 at which the stent is disposed). Preferably, in this embodiment, the proximal-side stopper 70 is a spring-formed stopper wound around the shaft section. As shown in FIGS. 5 and 9, the proximal-side stopper 70 includes a proximal-side coil section 70a wound around the distal tube 31, and a distal-side coil section 70b which extends in the distal direction from the proximal-side coil section 70a and which has a part not in contact with the distal tube 31. The distal-side coil section 70b in this embodiment is eccentrically fixed to the distal tube 31, and has both a part in contact with the distal tube 31 and a part separate (spaced) from the distal tube 31. In addition, the stent proximal portion fixing wire 5 penetrates (extends into) that part of the distal-side coil section 70b which is not in contact with the distal tube 31. A part of the wire 5 extending in the direction of the stent from one end part 5a of the wire may be fixed to the above-mentioned distal-side coil section 70b. The fixation of the wire 5 to the distal-side coil section 70b is preferably achieved by clamping between the turns of the coil. In addition, the distal-side coil section 70b functions as a stopper for the stent 10. Further, the distal-side coil section 70b may be so configured that substantially the whole part thereof is separate from the distal tube 31, as in the embodiment shown in FIG. 14.

The proximal-side coil section 70a is spring-formed, and can lock a proximal portion of the stent without damaging the stent. In addition, the stopper 70 may be formed of a radiopaque material. This allows the position of the vicinity of the proximal end of the stent to be visually observed or determined under radioscopy, thus facilitating the procedure. Preferable examples of the radiopaque material include gold, platinum, platinum-iridium alloy, silver, stainless steel, platinum, their alloys, etc. The stopper 70 is formed by forming a wire from the radiopaque material and winding the wire around the outer surface of the distal tube 31.

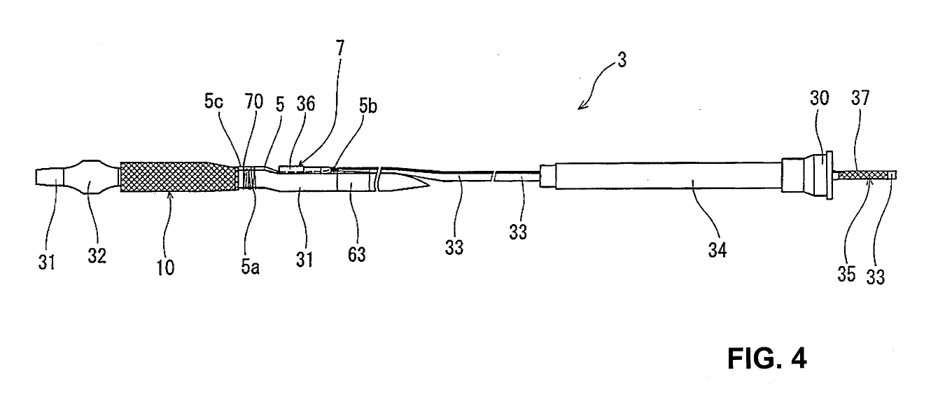

As shown in FIGS. 4-6 and 9, the shaft section 3 is outfitted with the heat-breaking stent proximal portion fixing wire 5 having one end portion (first portion) 5a and the other end portion (second portion) 5b fixed to the shaft section and having the intermediate portion (third portion) 5c engaged with a proximal portion of the stent 10, and the heat-breaking member 7 for breaking the stent proximal portion fixing wire 5 to release the stent 10 from the engagement.

In this embodiment, as shown in FIG. 9, the stent 10 used is a stent which has a plurality of through holes 18 through which can pass the stent proximal portion fixing wire. The holes 18 are provided in proximal-side joint sections 16 of the stent and are arranged in a substantially annular pattern. Generally speaking, the proximal-side joint sections 16 constitute connection members at the proximal end portion of the stent that allow the stent to be connected to the stent proximal portion fixing wire 5. The through holes 18 constitute one example of openings at the proximal end of the stent allowing the fixing wire 5 to engage the stent. The intermediate portion 5c of the stent proximal portion fixing wire 5 penetrates sequentially the plurality of through holes 18 of the stent 10, and passes through the plurality of through holes 18 in an annular pattern as a whole. Therefore, the stent 10 is engaged with (fixed to) the shaft section 3 by the stent proximal portion fixing wire 5, and would not be released from the shaft section 3 unless the stent proximal portion fixing wire 5 is broken (cut).

In this embodiment, one end portion 5a of the stent proximal portion fixing wire 5 is wound around the outer surface of the distal tube 31 and is fixed there by an adhesive 51, in the vicinity of and slightly proximally relative to the stopper 70. In addition, the other end portion 5b of the stent proximal portion fixing wire 5 is wound around the outer surface of the shaft body 33 and is fixed to the distal end portion of the shaft body 33. The one end portion 5a and the other end portion 5b of the stent proximal portion fixing wire 5 are not limited to being wound around and fixed to the outer surfaces of the distal tube 31 and the shaft body 33, respectively. The one end portion 5a and the other end portion 5b of the stent proximal portion fixing wire 5 may be fixed respectively to the outer surfaces of the distal tube 31 and the shaft body 33 by caulking rings. Further, in this embodiment, the stent proximal portion fixing wire 5 extends in the direction of the stent by passing through the gaps in the coil constituting the spring-formed stopper 70, from one end portion 5a and the other end portion 5b which are fixed to the shaft section. Specifically, the stent proximal portion fixing wire 5 at the part extending from one end portion 5a and the stent proximal portion fixing wire 5 at the part extending from the other end portion 5b are both extending by passing over the proximal-side coil section 70a of the stopper 70 and penetrating between the distal-side coil section 70b and the distal tube 31. With the stopper formed in this manner, it exhibits an effect as a stopper for the stent proximal portion, and guides (penetrates) the stent fixing wire, whereby fixation of the stent by the wire is relatively assured. In addition, at the time of releasing the wire from the stent, the wire is prevented from be entangled on the stent, to facilitate release.

The heat-breaking stent proximal portion fixing wire 5 is preferably a thermoplastic resin fiber. The thermoplastic resin is preferably a synthetic resin such as polyethylene, polypropylene, nylon, polyethylene terephthalate, etc., particularly, one that has a low melting point. The heat-breaking stent proximal portion fixing wire may have a configuration in which only its portion near the part to be heat-broken is formed of a low-melting-point resin. The heat-breaking stent proximal portion fixing wire may be composed of a single thermoplastic resin fiber, a plurality of thermoplastic resin fibers bundled together or twisted together, or the like.

The shaft section 3 has the heat-breaking member 7 for breaking the stent proximal portion fixing wire 5 to release the stent 10 from the engagement condition. In this embodiment, the heat-breaking member 7 includes a heat generating section (heating unit) 36 for breaking, electric cables 64, 65 having distal portions connected to the heat generating section 36 and extending to a proximal portion of the shaft body 33, and a joint section 35 to be joined to a power supply device. The joint section 35 is at a proximal portion of the shaft body 33. In this embodiment, the heat generating section 36 for breaking the heat-breaking member 7 is fixed to the distal end of the shaft body 33, and the electric cables 64, 65 extend to the proximal portion of the shaft body 33 and are fixed to the outer surface of the shaft body 33.

The joint section 35 for joining to the power supply device is at the proximal portion of the shaft body 33. The joint section 35 is formed at the outer surface of the proximal potion of the shaft body 33, and includes a first electrode part 37 electrically connected to the cable 64, and a second electrode 38 connected to the cable 65. In this embodiment, an insulating part 39 provides insulation between the first electrode 37 and the second electrode 38. A part of the heat-breaking stent proximal portion fixing wire 5, in this embodiment, a part spaced a predetermined distance toward an intermediate part side relative to the other end portion 5b, is enveloped by the heat generating section 36 for breaking. By virtue of electric power supplied to the first electrode part 37 and the second electrode 38 of the joint section 35, heat is generated in the heat generating section 36 for breaking, whereby the heat-breaking stent proximal portion fixing wire 5 is broken through fusion at the position of the heat generating section 36.

The stent 10 used in the stent delivery system here is a so-called self-expandable stent which is capable of expanding (automatically expanding) outward when placed (put indwelling) in a living body so that the stent is restored into its pre-compression shape. The stent 10 includes a distal portion oriented toward the distal side of the sheath 2 and a proximal portion oriented toward the proximal side of the sheath 2. Also, other than the proximal end portion of the stent 10 at which the proximal-most end portions 13a, 14a are joined together at the joint portions 16 as shown in FIG. 11, the stent 10 is devoid of bent free ends projecting toward the proximal side (in the proximal direction). By moving the sheath 2 after exposure of a distal portion of the stent 10 from the sheath 2, the exposed distal portion can be re-contained in the sheath 2.

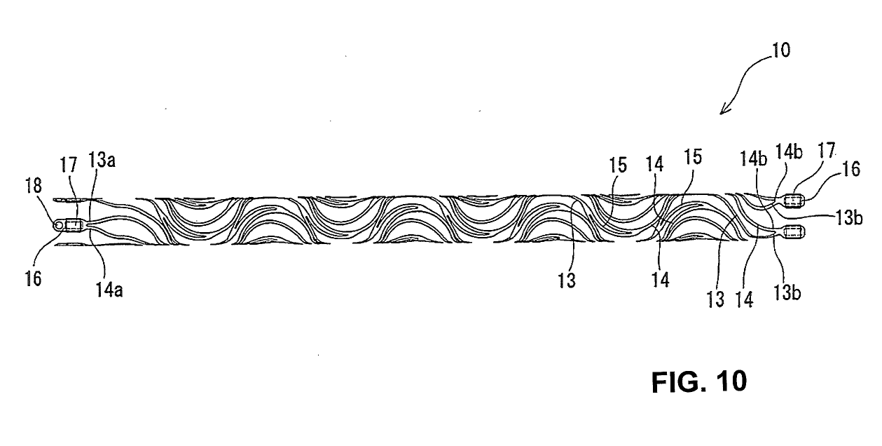

The stent to be used may be a stent as shown in FIGS. 10 and 11.

This illustrated stent 10 includes a plurality of wave-shaped struts 13, 14 extending in the axial direction of the stent from one end side to the other end side of the stent and arranged in the circumferential direction of the stent, and one or more connecting struts 15 which each interconnect the wave-shaped struts which are adjacent to each other. The connecting struts 15 extend in the axial direction over a predetermined length. The end portions of each wave-shaped strut 13, 14 are respectively joined to the end portion of the circumferentially adjacent wave-shaped strut.

To describe in slightly more detail, the stent 10 shown in FIGS. 10 and 11 includes: a plurality of first wave-shaped struts 13 extending in the axial direction of the stent 10 from one end side to the other end side of the stent 10 and arranged in the circumferential direction of the stent; a plurality of second wave-shaped struts 14 located between the first wave-shaped struts 13, extending in the axial direction of the stent from one end side to the other end side of the stent, and arranged in the circumferential direction of the stent; one or more connecting struts 15 which each interconnect the first wave-shaped strut 13 and the second wave-shaped strut 14 which are adjacent to each other, and with the one or more connecting struts 15 extending in the axial direction over a predetermined length. Apexes of the second wave-shaped struts 14 are shifted a predetermined distance in the axial direction of the stent relative to those apexes of the first wave-shaped struts 13 which are close to the apexes of the second wave-shaped struts 14 in the circumferential direction of the stent 10 and are curved in the same direction as the apexes of the second wave-shaped struts 14. In addition, the first wave-shaped strut 13 has end portions 13a, 13b joined to end portions 14a, 14b of the circumferentially adjacent second wave-shaped strut.

The stent 10 in this embodiment is a so-called self-expandable stent which is formed in a substantially cylindrical shape, is compressed toward its central axis at the time of insertion into a living body, and expands outward when placed (put indwelling) in the living body, to be restored into its pre-compression shape.

The first wave-shaped struts 13 extend in the axial direction substantially parallel to the central axis of the stent. A plurality of the first wave-shaped struts 13 are arranged in the circumferential direction of the stent. The number of the first wave-shaped struts 13 is preferably three or more, particularly about three to eight. Further, the plurality of the first wave-shaped struts 13 are preferably so arranged that they are each arranged at the same angle (inclusive of substantially the same angle) relative to the central axis of the stent.

The second wave-shaped struts 14 also extend in the axial direction substantially parallel to the center axis of the stent. A plurality of the second wave-shaped struts 14 are arranged in the circumferential direction of the stent, and each of the second wave-shaped struts 14 is disposed between the first wave-shaped struts. The number of second wave-shaped struts 14 is preferably three or more, particularly about three to eight. Further, the plurality of the second wave-shaped struts 14 are preferably so arranged that they are each arranged at the same angle (inclusive of substantially the same angle) relative to the central axis of the stent. The number of second wave-shaped struts 14 is the same as the number of first wave-shaped struts 13.

The stent 10 has one or more connecting struts 15 each of which interconnects the first wave-shaped strut 13 and the second wave-shaped strut 14 circumferentially adjacent to each other and which extend in the axial direction over a predetermined length. More specifically, in the stent 10 in this embodiment, the connecting struts 15 possess one end near an inflection point of the wave-shaped strut on one side, and possess the other end in a region ranging from a position near an apex of that wave-shaped strut on the other side which is adjacent to the wave-shaped strut on one side to a position slightly beyond the apex. The connecting struts 15 extend in the axial direction, and is curved in the same direction as the apex of the wave-shaped strut on the other side. Specifically, as shown in FIG. 11, the connecting struts 15 are composed of first connecting struts 15a which have apexes directed to one side in the circumferential direction of the stent 10 and are curved, and second connecting struts 15b which have apexes directed to the other side in the circumferential direction of the stent 10 and are curved. The connecting struts 15 are curved in a circular arc shape, and have a radius approximately equal to the radius of the circular arc shape of the curved part of the first wave-shaped strut 13 or the second wave-shaped strut 14 close to the connecting struts in the circumferential direction of the stent 10.

The stent 10 in this embodiment includes the joint sections 16 by which every one of the one-end-side end portions and the-other-end-side end portions of all the first wave-shaped struts is joined to an end portion of either one of the second wave-shaped struts close to the first wave-shaped strut. Specifically, one-end-side end portion 13a of the first wave-shaped strut of the stent 10 is joined to one-end-side end portion 14a of that second wave-shaped strut 14 on one side which is circumferentially adjacent to the first wave-shaped strut (specifically, that second wave-shaped strut 14 which is located close to and on the other side in the circumferential direction of the first wave-shaped strut) by the joint section 16. In addition, the-other-end-side end portion 13b of the first wave-shaped strut is joined to the-other-end-side end portion 14b of that second wave-shaped strut 14 on one side which is circumferentially adjacent to the first wave-shaped strut (specifically, that second wave-shaped strut 14 which is located close to and on one side in the circumferential direction of the first wave-shaped strut) by the joint section 16. In other words, the joint section 16 on one end side and the joint section 16 on the other end side are different from each other (successively shifted in the circumferential direction so that they are not axially aligned) in combination of the first wave-shaped strut 13 and the second wave-shaped strut 14 which are joined to each other.

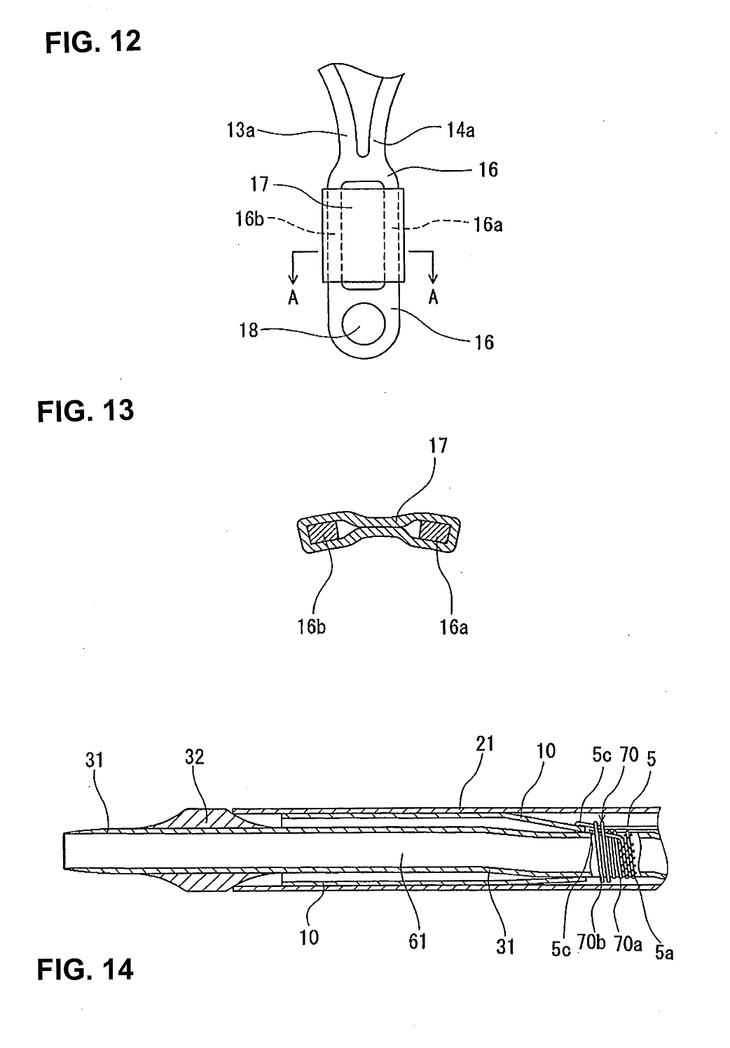

The joint sections 16 are each fitted with a radiopaque marker 17, as shown in FIGS. 10-12. In this embodiment, as shown in FIG. 12, the joint section 16 has two frame parts 16a, 16b extending toward the end portion parallel to each other with a predetermined spacing therebetween, and the radiopaque marker 17 envelopes the two frame parts 16a, 16b entirely (inclusive of substantially entirely) or partly. In addition, the radiopaque marker 17 is in the shape of a thin-walled rectangular parallelepiped, accommodates or encloses the two frame parts 16a, 16b, and is recessed in a central area so as to be fixed to the two frame parts 16a, 16b. Examples of the material which can be suitably used to form the radiopaque marker include one (elemental substance) or two or more (alloy) selected from the group consisting of iridium, platinum, gold, rhenium, tungsten, palladium, rhodium, tantalum, silver, ruthenium, and hafnium.

The stent 10 is provided, in each of the joint sections 16 on the proximal side portion, with a through hole 18 (relatively small hole) through which passes the stent proximal portion fixing wire. The through holes 18 extend in the direction of the center of the stent. The through holes 18 permitting passage of the stent proximal portion fixing wire preferably have a low-friction inner surface or a relatively easily releasable form for enhancing releasability of the wire 5 from the through holes. The low-friction inner surface can be formed by making the inner surface a smooth surface, or coating the inner surface with a low-friction material, or a similar method.

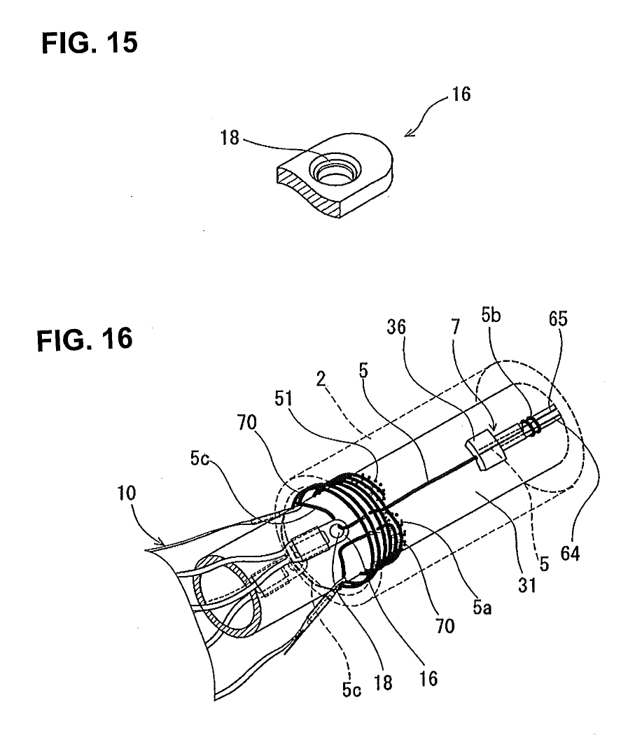

An example of a relatively easily releasable form of the through holes is shown in FIG. 15. The illustrated through hole 18 formed in the joint section 16 has a form in which the edge of the opening of the small hole 18 is chamfered or enlarged in diameter in a tapered form. The through hole 18 may be chamfered or enlarged in diameter in a tapered form, at both the edges of the opening on the outer surface side and the inner surface side of the stent. This helps facilitate relatively easy passage and release of the stent fixing wire.

The material constituting the stent 10 is preferably a superelastic metal. As the superelastic metal, superelastic alloys are preferably used. Superelastic alloys means those alloys which are generally called shape memory alloys and which show superelasticity at least at a living body temperature (around 37° C.). Particularly preferable for use here are superelastic metal bodies such as Ti—Ni alloys containing 49 to 53 atomic % of Ni, Cu—Zn alloys containing 38.5 to 41.5 wt % of Zn, Cu—Zn—X alloys (X═Be, Si, Sn, Al, Ga) containing 1 to 10 wt % of X, Ni—Al alloys containing 36 to 38 atomic % of Al, etc. Especially preferred are the above-mentioned Ti—Ni alloys. Besides, use of Ti—Ni—X alloys (X=Co, Fe, Mn, Cr, V, Al, Nb, W, B or the like) prepared by replacing part of the Ti—Ni alloys by 0.01 to 10.0% of X, use of Ti—Ni—X alloys (X=Cu, Pb, Zr) prepared by replacing part of the Ti—Ni alloys by 0.01 to 30.0% of atoms, or selection of cold working ratio and/or final heat treatment conditions, may be made, whereby mechanical properties of the superelastic alloy can be changed, as required. While using the above-mentioned Ti—Ni—X alloy, the cold working ratio and/or final heat treatment conditions may be selected, whereby the mechanical properties of the alloy can be changed, as required. Of the superelastic alloy to be used, the buckling strength (the yield stress under load) is 5 to 200 kg/mm² (22° C.), preferably 8 to 150 kg/mm², and the restoring stress (the yield stress when unloaded) is 3 to 180 kg/mm² (22° C.), preferably 5 to 130 kg/mm². The superelasticity here means a property of a metal such that even upon deformation (bending, extension, compression) of the metal into a region where ordinary metals undergo plastic deformation at use temperature, the deformed metal is restored substantially into its pre-compression shape after release of the deformation, without needing heating.

The diameter of the stent in a compressed state is preferably about 0.5 to 1.8 mm, more preferably 0.6 to 1.4 mm. The length of the stent in a non-compressed state is preferably about 5 to 200 mm, more preferably 8.0 to 100.0 mm. The diameter of the stent in a non-compressed state is preferably about 1.5 to 6.0 mm, more preferably 2.0 to 5.0 mm. Additionally, the material thickness of the stent is preferably about 0.05 to 0.40 mm, more preferably 0.05 to 0.15 mm, and the width of the wave-shaped struts is preferably 0.01 to 1.00 mm, more preferably 0.05 to 0.2 mm. Surfaces of the wave-shaped struts are preferably processed to be smooth. In this embodiment smoothening is preferably carried out by electropolishing. The strength of the stent in the radial direction is preferably 0.1 to 30.0 N/cm, more preferably 0.5 to 5.0 N/cm.

The operation of the stent delivery system described above is now set forth with reference to FIGS. 9 and 16-18.

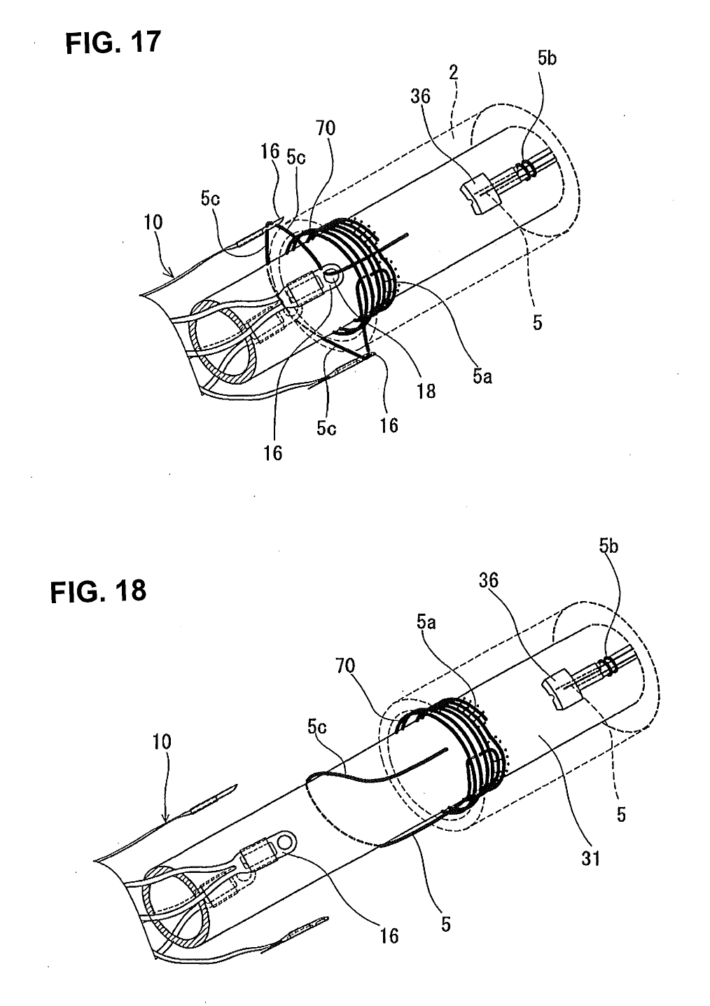

When the entirety of the stent 10 is contained in the sheath 2, the stent delivery system is in the condition shown in FIG. 9. The stent delivery system is manipulated to move the distal portion of the stent delivery system to a desired position in a living body lumen to deliver the stent at the desired location. Then, sliding the sheath 2 in the proximal direction toward the proximal side as shown in FIG. 16 causes the stent 10 to be exposed from the distal opening of the sheath 2 so that the stent is exposed distally beyond the distal end of the sheath 2. The stent 10 exposed from the sheath 2 exhibits a tendency to expand by its self-expanding force so as to be restored toward its pre-compression state. In this stent delivery system, however, the proximal portion of the stent 10 is (remains) engaged with the shaft section 3 by the heat-breaking stent proximal portion fixing wire 5 so that the proximal portion of the stent 10 cannot expand, and stays in the state shown in FIG. 16. In the case where it is necessary to re-adjust the position of the stent 10, the stent 10 can be re-contained into the sheath by sliding the sheath 2 in the distal direction. After it is confirmed that the stent 10 is disposed at a target lesion, the power supply device (not shown) connected to the shaft section 3 is operated to cause the heat generating section 36 for breaking to generate heat, thereby breaking the stent proximal portion fixing wire 5. As a result, the proximal portion of the stent 10 is released from the engagement by the heat-breaking stent proximal portion fixing wire 5, and the proximal portion of the stent 10 also expands as shown in FIG. 17. Thereafter, the stent delivery system 1 (the sheath 2 and the shaft section 3) in which the stent is released is moved in the proximal direction, whereby the intermediate portion 5c of the stent proximal portion fixing wire 5 having held the stent 10 in engagement is released from the stent as shown in FIG. 18. The broken stent proximal portion fixing wire 5, inclusive of the intermediate portion 5c, has one end that remains fixed to the shaft section 3, and so the wire 5 is neither discharged into the living body nor left remaining in the stent.

The detailed description above describes features and aspects of an embodiment of the stent delivery system disclosed here. The invention is not limited, however, to the precise embodiment and variations described. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. A stent delivery system comprising:
a shaft section through which extends a guide wire lumen which is open at opposite ends for receiving a guide wire to guide the stent delivery system to a desired position within a living body;
a sheath encircling at least a distal portion of the shaft section so that an inner surface of the sheath faces an outer surface of the shaft section, with a space between the inner surface of the sheath and the outer surface of the shaft section, the sheath being movable in a proximal direction relative to the shaft section;
a self-expandable stent possessing a central axis and a proximal end, the stent encircling the distal portion of the shaft section and being positioned in the space between the inner surface of the sheath and the outer surface of the shaft section so that the sheath covers the stent;
the stent being compressed inwardly towards the central axis of the stent while covered by the sheath, and automatically expanding outwardly into a pre-compression shape when the sheath is moved in the proximal direction relative to the shaft section to a position no longer covering the stent;
the stent comprising a plurality of through openings at a proximal end of the stent;
a breakable fixing wire comprising a first portion and a second portion both fixed to the shaft section so that the first portion and the second portion of the breakable fixing wire move together as a unit with the shaft section, the breakable fixing wire comprising a third portion spaced from the first portion and the second portion, the third portion of the breakable fixing wire passing through the through openings at the proximal end of the stent so that the breakable fixing wire engages the stent and holds the stent relative to the shaft section; and
means for breaking the fixing wire to release the stent from engagement by the other portion of the fixing wire, the third portion of the fixing wire being a portion of the fixing wire between the first portion and the second portion.

2. The stent delivery system according to claim 1, wherein the means for breaking is a heating unit which generates heat.

3. A stent delivery system that delivers a stent to a desired position in a living body, the stent delivery system comprising:
a cylindrically shaped stent possessing a central axis, the stent being compressed toward the central axis of the stent at the time of insertion into a living body, the stent being configured to automatically expand outwardly when placed in the living body to be restored into a pre-compression shape;
a shaft section having a guide wire lumen, the shaft section possessing a distal end and a proximal end;
a sheath containing the stent, the stent being located on the shaft section at a position near the distal end of the shaft section;
a stent proximal portion fixing wire possessing one end portion and an other end portion, the one end portion and the other end portion of the stent proximal portion fixing wire being fixed to the shaft section;
the stent proximal portion fixing wire also possessing an intermediate portion which is in engagement with a proximal portion of the stent; and
a breaking member for breaking the stent proximal portion fixing wire to release the stent from engagement by the intermediate portion of the stent proximal portion fixing wire.

4. The stent delivery system according to claim 3, wherein the proximal portion of the stent includes a plurality of through holes arranged in an annular pattern, the intermediate portion of the stent proximal portion fixing wire passing through the plurality of through holes.

5. The stent delivery system according to claim 3, wherein the shaft section comprises a shaft body and a distal tube, the distal tube being provided with the guide wire lumen, the shaft body having a distal portion fixed to a proximal end portion of the distal tube, and the breaking member is secured to the distal portion of the shaft body.

6. The stent delivery system according to claim 3, wherein the stent has a distal portion oriented toward a distal side of the sheath and the proximal portion oriented toward a proximal side of the sheath, the stent being devoid of any free bent end projecting toward the proximal side, other than at a proximal-most end portion of the stent, the sheath being movable in a proximal direction relative to the shaft section to expose the distal portion of the stent, the sheath being movable in a distal direction relative to the shaft section after exposing the distal portion of the stent so that the distal portion of the stent is once again covered by the sheath.

7. The stent delivery system according to claim 3, wherein the stent proximal portion fixing wire is breakable upon application of heat, and the breaking member comprises a heating unit which generates heat.

8. The stent delivery system according to claim 7, wherein the heating unit is connected to electric cables fixed to an outer surface of the shaft section, the electric cables being connected to a joint section at a proximal portion of the shaft section.

9. The stent delivery system according to claim 3, wherein the shaft section has a proximal-side opening communicating with the guide wire lumen and through which the guide wire is adapted to be passed, wherein the proximal-side opening opens to a side portion of the shaft section at a position spaced from distal and proximal ends of the shaft section, the proximal-side opening being located proximally of a proximal-most end of the stent.

10. The stent delivery system according to claim 3, wherein the sheath is proximally movable relative to the shaft section to expose the stent and allow the stent to automatically expand outwardly, the sheath being distally movable relative to the shaft section after the stent has automatically expanded outwardly to once again cover the stent until the stent is released from the engagement by the intermediate portion of the stent proximal portion fixing wire.

11. The stent delivery system according to claim 3, wherein the stent proximal portion fixing wire is a thermoplastic resin fiber.

12. The stent delivery system according to claim 3, further comprising stopper secured to the shaft section to restrain the stent from moving in a proximal direction, the stopper being positioned proximally of the proximally relative to the proximal end of the stent.

13. The stent delivery system according to claim 12, wherein the stopper is a spring wound around the shaft section.

14. The stent delivery system according to claim 13, wherein the stent proximal portion fixing wire extends, from the one end portion and the other end portion, in a distal direction toward the stent by passing through a gap in a coil constituting the spring.

15. The stent delivery system according to claim 3, wherein the proximal portion of the stent includes a plurality of through holes arranged in an annular pattern, the intermediate portion of the stent proximal portion fixing wire passing through the plurality of through holes, each of the through holes possessing one of a low-friction inner surface or a tapered inner diameter to facilitate release of the stent proximal portion fixing wire.

16. The stent delivery system according to claim 3, wherein the stent comprises a plurality of wave-shaped struts extending in a axial direction of the stent from one axial end to an opposite axial end of the stent, the wave-shaped struts being circumferentially arranged and spaced apart from one another, the stent also comprising connecting struts each interconnecting circumferentially adjacent wave-shaped struts, the connecting struts extending in the axial direction over a predetermined length shorter than the wave-shaped struts, the wave-shaped struts each having end portions, each end portion of each wave-shaped strut being connected to the end portion of the circumferentially adjacent wave-shaped strut.

17. The stent delivery system according to claim 16, wherein the connecting struts are each curved in a circular arc shape.

18. The stent delivery system according to claim 16, wherein each end portion of each wave-shaped strut is connected to the end portion of the circumferentially adjacent wave-shaped strut at a joint section, and wherein a through hole extends through each of the joint sections to thus provide a plurality of through holes arranged in an annular pattern, the intermediate portion of the stent proximal portion fixing wire passing through the plurality of through holes.

19. A method of operating a stent delivery system to deliver a stent to a desired position in a living body, the method comprising:
positioning a distal portion of a stent delivery system at a desired location in a living body lumen, the stent delivery system comprising: a shaft section; a sheath surrounding a distal portion of the shaft section with an annular space between the shaft section and the sheath; a self-expandable stent surrounding the distal portion of the shaft section, positioned in the space, and covered by the sheath, the stent being compressed toward a central axis of the stent and configured to automatically expand outwardly when the sheath is moved in a proximal direction relative to the shaft section to expose the stent; and a stent proximal portion fixing wire having one end portion fixed to the shaft section and an other portion in engagement with a proximal portion of the stent;
proximally moving the sheath relative to the stent to uncover the stent and allow the stent to automatically expand outwardly, except for the proximal portion of the stent which continues to be engaged by the other portion of the stent proximal portion fixing wire in a manner preventing the proximal portion of the stent from expanding outwardly; and
breaking the stent proximal portion fixing wire to release the proximal portion of the stent and allow the proximal portion of the stent to automatically expand outwardly.

20. The method according to claim 19, further comprising, after proximally moving the sheath to uncover the stent and before breaking the stent proximal portion fixing wire, distally moving the sheath to a position in which the sheath once again covers the stent.
